(19) United States
(12) Patent Application Publication
(10) Pub. No.: US 2014/0276535 A1
(43) Pub. Date: Sep. 18, 2014

(54) ELECTRONIC NEEDLELESS INJECTOR

(71) Applicants: Michael Wood, Carthage, IL (US); Chris Vanden Berghe, Carthage, IL (US)

(72) Inventors: Michael Wood, Carthage, IL (US); Chris Vanden Berghe, Carthage, IL (US)

(21) Appl. No.: 13/828,091

(22) Filed: Mar. 14, 2013

(51) Int. Cl. A61M 5/30

(52) U.S. Cl. CPC A61M 5/30
USPC 604/500; 604/68

(57) ABSTRACT

A needleless injector includes an injector body, a nozzle at one end of the body, a medicine inlet for receiving medicine into the injector body, a rod disposed within the injector body for driving the medicine towards the nozzle, and a sensor for monitoring operation of the needleless injector. The sensor may be a position sensor. The sensor may be a slide potentiometer. The sensor may provide for monitoring firing of the rod and/or monitoring length of a stroke of the rod. An intelligent control operatively may be operatively connected to the sensor. An RFID reader may also be operatively connected to the intelligent control for associating RFID data with operations of the needleless injector. A keypad may be operatively connected to the intelligent control and a display may be operatively connected to the intelligent control.

### FIELD OF THE INVENTION

The present invention relates to a needleless injector and its use. More specifically, but not exclusively, the present invention relates to an electronic needleless injector.

### BACKGROUND OF THE INVENTION

Vaccination or administration of medicine by injection has long been performed by using syringes with needles. However, there are problems with this method. Needles can cause damage which can be problematic as it may adversely affect the grading of an animal and result in financial loss for meat producers. Moreover, use of needles risks needles breaking in the animal resulting in food safety issues.

Although it is known to use a needleless injector, various problems exist with various approaches to needleless injection. In addition, there are various reasons why needleless injector operations may fail resulting in animals that do not benefit from the injections and incomplete or faulty records of injections. What is needed is an electronic needleless injector.

### SUMMARY OF THE INVENTION

Therefore, it is a primary object, feature, or advantage of the present invention to improve over the state of the art.

It is a further object, feature, or advantage of the present invention to provide an electronic needleless injector.

It is another object, feature, or advantage of the present invention to provide a needleless injector which is capable of monitoring the injection operation.

It is another object, feature, or advantage of the present invention to provide a needleless injector which is capable of data capture.

A still further object, feature, or advantage of the present invention is to provide a needleless injector which may be pneumatically controlled.

Another object, feature, or advantage of the present invention is to provide for associating individual animals with injection operations.

Yet another object, feature, or advantage of the present invention is to improve food safety.

A still further object, feature, or advantage of the present invention is to improve traceability of animal products.

One or more of these and/or other objects, features, or advantages of the present invention will become apparent from the specification and claims that follow. Different embodiments may have different objects, features, or advantages.

According to one aspect, a needleless injector is provided. The needleless injector includes an injector body, a nozzle at one end of the body, a medicine inlet for receiving medicine into the injector body, a rod disposed within the injector body for driving the medicine towards the nozzle, and a sensor for monitoring operation of the needleless injector. The sensor may be a position sensor. The sensor may be a slide potentiometer. The sensor may provide for monitoring firing of the rod and/or monitoring length of a stroke of the rod. An intelligent control operatively may be operatively connected to the sensor. An RFID reader may also be operatively connected to the intelligent control for associating RFID data with operations of the needleless injector. A keypad may be operatively connected to the intelligent control and a display may be operatively connected to the intelligent control.

According to another aspect, a method for collecting data associated with operation of a needleless injector is provided. The method includes providing a needleless injector comprising an injector body, a nozzle at one end of the body, a medicine inlet for receiving medicine into the injector body, a rod disposed within the injector body for driving the medicine towards the nozzle, and a sensor for monitoring operation of the needleless injector. The method further includes monitoring operation of the needleless injector using the sensor to provide sensor data, communicating the sensor data to an intelligent control, and reporting on operation of needleless injector to a user.

### BRIEF DESCRIPTION OF THE DRAWINGS

FIG. 1 is a block diagram illustrating an example of the invention.

FIG. 2A illustrates an injector body with a rod in an initial position.

FIG. 2B illustrates an injector body with a rod in a partially extended position.

FIG. 2C illustrates an injector with a rod in an extended position.

### DETAILED DESCRIPTION OF THE PREFERRED EMBODIMENT

The present invention provides for an electronic needleless injector. The electronic needleless injector may have electronic control, electronic sensing, and/or electronic feedback.

FIG. 1 illustrates one embodiment of a system including a needleless injector. The system 10 includes a needleless injector 12. The needleless injector is configured to needlelessly inject medicine in fluid form into an animal. A medicine container 14 is shown which is fluidly connected to the needleless injector. Although shown separately, it is contemplated that in some embodiments the medicine container 14 may be integrated into the needleless injector, however, it may be preferred to keep it separate in order to reduce size of the needleless injector 12.

The needleless injector 12 includes one or more sensors 16. The one or more sensors 16 are used for monitoring operation of the needleless injector 12. The one or more operations can include determining whether medicine was successfully delivered, whether the needleless injector operated properly, whether medicine was not delivered, whether the needleless injector did not operate properly or otherwise monitor operation of the needleless injector as is explained in greater detail herein. The one or more sensors 12 are operatively connected to an intelligent control 20. The intelligent control may be within the needleless injector or may be separate from the body of the needleless injector and may communicate through a wired connection or wirelessly with electronics within the needleless injector 12. For example, the sensor(s) 16 may be operatively connected to a transceiver 34 in the grip of the injector 12 which communicates information to a transceiver 36 which is operatively connected to the intelligent control 20. The intelligent control 20 may also be operatively connected to a display 22. The display 22 may be used for displaying information about an injection or information otherwise associated with an injection. The intelligent control 20 may also be operatively connected to a keypad 24 which may be used to provide for receiving user input from a user. The user input may include setting information for the needleless injector. The user input may include information regarding an individual who is operating the needleless injector. The user input may include information regarding an animal which is receiving an injection.

An RFID scanner 26 is also operatively connected to the intelligent control 20. The RFID scanner may be used to scan an RF tag associated with an animal, a person (such as a person administering an injection), a location (such as a particular building or facility), or supplies (such as a tag associated with a container of medicine). Using an RFID scanner and RFID tags assists in automating the process of data collection. Automating the process of data collection may be advantageous in various ways. First, it makes the process of data collection more convenient for users. Second, it generally provides for more reliable data collection. In addition, to the convenience and reliability of the data collection it makes it viable to collect information which may not otherwise normally be collected.

The availability of data which is reliably collected can be important for numerous reasons. This includes record-keeping for an animal production operation. In addition, the record-keeping may be advantageous for records relating to meat traceability and food safety. So, for example, a determination can be made as to what type of injection was given to a particular animal, when the injection was given, by whom the injection was given, whether there were any issues with the injection, or other information.

Instead of an RFID scanner 26, other types of electronic identifying technologies may be used as appropriate. In addition, data may be manually input instead. For example, the keypad 24 may be used to enter identifying information such as an identifier on an ear tag or tattoo, or other type of tag associated with an animal. It is also contemplated, that instead of or in addition to an identifier unique to an animal, a pen number, building number, or other identifier may be used to indicate a group of animals or location of animals.

Data storage 28 is operatively connected to the intelligent control 20. The data storage may be in the form of a machine readable storage medium. The machine readable storage medium may be used for storing information associated with operation of the injector including information based on the one or more sensors, information entered through the keypad, received using the RFID scanner, or other information.

An interface 30 is also operatively connected to the intelligent control 20. The interface 30 may be of any number of wired or wireless types of interfaces. The interface 30 may, for example, be a USB interface or other type of network interface used for communications between the system 10 and other types of computing devices such as notebook computers, tablet computers, phones, desktop computers, or other devices.

A clock 32 is also shown which is operatively connected to the intelligent control 20. The clock 32 may be used for various purposes. It may, for example, be used to maintain time and date information so that sensor readings may be time and date stamped. Alternatively, or in addition, to that function the clock 32 may be used for timing different operations associated with operation of the injector in order to assist in determining if the injector is operating properly.

Although various components are shown in FIG. 1 separately, it is to be understood that various components may be integrated together. For example, the intelligent control 20 may be a microcontroller with data storage 28 and the clock 32 integrated therewith.

In addition, as shown in FIG. 1, valving 38 may be operatively connected to the intelligent control 20 either wirelessly through transceivers 34, 36 or through a wired connection. The intelligent control 20 may be configured to control the valving 38 in order to adjust parameters such as dosage or other parameters that may be electronically controlled.

FIGS. 2A-2C illustrate on example of using a sensor. In FIG. 2A-2C the injector 12 is shown with a housing 40. A medicine inlet 50 is shown for receiving medicine. An injector body 44 is disposed within the housing with a rod 42. Movement of the rod 42 is sensed with sensor 44 which is a potentiometer. Thus, as the rod 42 transitions between different positions during delivery of medicine with the rod through chamber 46 and nozzle 48, the potentiometer 44 senses the position of the rod 42. FIG. 2A illustrates a first position where the rod is 0 percent extended. FIG. 2B illustrates an intermediate position where the rod is partially extended. FIG. 2C illustrates a fully extended position where the rod is 100 percent extended.

By monitoring the position of the sensor 44 during an injection cycle one can determine whether the injection cycle was completed and medicine was delivered or not. In addition to determining stroke of the rod 42 the amount of time associated with the stroke can also be measured. The amount of time can provide additional insight into the operation of the injector 12.

Returning to FIG. 1, where the sensor(s) 16 include the potentiometer configured in the manner shown in FIG. 2A-2C, the intelligent control can determine a stroke associated with the rod which may be measured as a percentage. This information may be displayed on the display 22, stored in data storage 28, and/or communicated over the interface 30. Such information may be used to determine whether the injection occurred under the right conditions or not. This can include determining whether or not there was medicine in the chamber at the time of injection, whether a complete injection cycle was completed, whether the timing of an injection cycle was consistent with proper operation.

Although various embodiments of an electronic needleless injector have been described, the present invention contemplates numerous other embodiments. This may include injectors with various types of configurations, various types of sensors, various types of data capture and reporting, and other options, variations, and alternatives.

What is claimed is:

1. A needleless injector, comprising:
an injector body;
a nozzle at one end of the body;
a medicine inlet for receiving medicine into the injector body;
a rod disposed within the injector body for driving the medicine towards the nozzle; and
a sensor for monitoring operation of the needleless injector.

2. The needleless injector of claim 1 wherein the sensor is a position sensor.

3. The needleless injector of claim 1 wherein the sensor comprises a slide potentiometer.

4. The needleless injector of claim 1 wherein the sensor provides for monitoring firing of the rod.

5. The needleless injector of claim 1 wherein the sensor provides for monitoring length of a stroke of the rod.

6. The needleless injector of claim 1 further comprising an intelligent control operatively connected to the sensor.

7. The needleless injector of claim 6 wherein the intelligent control is adapted to interpret data from the sensor and provide an output indicative of operation of the needleless injector.

8. The needleless injector of claim 7 further comprising an RFID reader operatively connected to the intelligent control for sensing RFID data and wherein the intelligent control is adapted for associating RFID data with operations of the needleless injector.

9. The needleless injector of claim 7 further comprising a keypad operatively connected to the intelligent control.

10. The needleless injector of claim 7 further comprising a display operatively connected to the intelligent control.

11. The needleless injector of claim 1 further comprising valving fluidly connected to the medicine inlet, wherein the valving is operatively connected to the intelligent control and the intelligent control is configured for controlling the valving to modify delivery parameters.

12. A method for collecting data associated with operation of a needleless injector, the method comprising:
providing a needleless injector comprising an injector body, a nozzle at one end of the body, a medicine inlet for receiving medicine into the injector body, a rod disposed within the injector body for driving the medicine towards the nozzle, and a sensor for monitoring operation of the needleless injector;
monitoring operation of the needleless injector using the sensor to provide sensor data;
communicating the sensor data to an intelligent control;
reporting on operation of needleless injector to a user.

13. The method of claim 12 further comprising storing the sensor data.

14. The method of claim 12 further comprising determining an identifier of an animal receiving an injection from the needleless injector.

15. The method of claim 14 further comprising associating the identifier of the animal with the sensor data for the injection from the needleless injector and storing both the identifier and the sensor data.

16. The method of claim 15 wherein the step of determining the identifier of the animal comprises receiving user input of the identifier.

17. The method of claim 16 wherein the user input is received at a keypad operatively connected to the intelligent control.

18. The method of claim 16 wherein the step of determining the identifier of the animal comprises reading an electronic identifier associated with the animal.

19. The method of claim 18 wherein the electronic identifier is on an RFID tag associated with the animal.
